(19) United States
(12) Patent Application Publication
(10) Pub. No.: US 2004/0265268 A1
(43) Pub. Date: Dec. 30, 2004

(54) Compositions and methods for skin rejuvenation and repair

(76) Inventor: Deepak Jain, San Diego, CA (US)

Correspondence Address: Biotechnology Law Group, 658 Marsolan Ave., Solana Beach, CA, 92075-1931, US

(21) Appl. No.: 10/821,427

(22) Filed: Apr. 9, 2004

Related U.S. Application Data
(63) Continuation-in-part of application No. 10/222,949, filed on Aug. 16, 2002.
(60) Provisional application No. 60/313,306, filed on Aug. 18, 2001.
(60) Provisional application No. 60/313,307, filed on Aug. 18, 2001.
(60) Provisional application No. 60/313,313, filed on Aug. 18, 2001.
(60) Provisional application No. 60/313,314, filed on Aug. 18, 2001.

(51) Int. Cl. A61K 38/19; A61K 31/728

(52) U.S. Cl. 424/85.1; 514/474; 435/404; 514/54

(57) ABSTRACT

The present invention provides compositions for the repair of mammalian skin. The compositions contain cell growth enhancers to increase the growth rate of skin cells, stimulators of cell growth enhancers, nutrients to support log phase growth of skin cells, cell protectors to protect growing cells and enhanced cellular activity, antioxidants to protect rejuvenated cells, extracellular matrix proteins, stimulators of extracellular matrix proteins, and penetration enhancers. The compositions of the present invention are effective for repairing and rejuvenating mammalian skin, such that aging skin treated with the compositions has a significant reduction in the number of fine lines and wrinkles in the skin. The compositions are also effective for promoting the healing of skin that has suffered a wound, such as a sunburn or abrasion, and for promoting the growth of hair on the scalp.

This application is a continuation-in-part of U.S. application Ser. No. 10/222,949, filed Aug. 16, 2002, which claims priority to U.S. Application Ser. No. 60/313,306 filed Aug. 18, 2001, and U.S. Application Ser. No. 60/313,307, filed Aug. 18, 2001, and U.S. Application Ser. No. 60/313,313, filed Aug. 18, 2001 and U.S. Application Ser. No. 60/313,314, filed Aug. 18, 2001, each of which is hereby incorporated by reference in their entireties including all Tables, Figures, and claims.

### FIELD OF THE INVENTION

The present invention relates to the fields of personal and topical skin care, cosmetics, cosmeceuticals, skin rejuvenation, skin anti-aging, skin repair, and wound healing.

### BACKGROUND OF THE INVENTION

The following discussion of the background of the invention is merely provided to aid the reader in understanding the invention and is not admitted to describe or constitute prior art to the present invention.

Skin care products in the market claim to rejuvenate, regenerate and repair skin through various additives included in cosmetic and cosmeceutical formulations but generally camouflage the signs of aging skin. Some products claim to rejuvenate skin cells with cosmeceutical additives such as vitamins, hydroxy acids, and botanical extracts. Many of these products make broad claims, usually without scientific basis. Vitamins and hydroxy acids primarily act as exfoliants and facilitate the shedding of the surface cells to produce younger looking skin (U.S. Pat. No. 5,547,988). But these products provide only a temporary solution and only produce a superficial effect. Botanical extracts and herbal components are popular additives claiming biological skin rejuvenation but no active components are defined in these products and results are varied at best (U.S. Pat. No. 6,036,966). Other products claim to regenerate skin with biological ingredients such as growth factors. But growth factors are large protein molecules and are unable to penetrate the epidermis of the skin. Furthermore, these products lack specific delivery systems for delivering the growth factors to reach the target skin cells and therefore do not penetrate the skin. Consequently, they fail to demonstrate any beneficial effects.

A more fundamental and comprehensive approach is needed for treating aging skin that is based on science and the biology of the skin. Skin aging is a natural phenomenon that occurs over time. The primary element responsible for accelerating skin aging is overexposure to the sun's harmful rays causing photo damage. Photoaging can be slowed with the use of sunscreens and avoidance of sun exposure. But the more important and complex causes of skin aging are biological and are caused by a slowing of the division rate of skin cells and defective cross-linking of collagen and elastin fibers in the skin. With age, the skin fails to regenerate itself as quickly as it used to, and shows common signs of aging in terms of tone and texture. Also, collagen and elastin fibers in the underlying layers of the skin, which provide the scaffolding for the surface layers, begin to weaken and deteriorate with age causing the skin to lose elasticity and form sags, fine lines, and wrinkles. Thus, from a biological standpoint, an effective plan for rejuvenating and repairing skin must address the rejuvenation of skin cells at both the epidermal and the dermal layers, protection of the rejuvenated cells and cellular activity, stimulation of the production of skin matrix elements, and the sustainability of the rejuvenated conditions over the long term. Biologically based components such as large molecular weight proteins are unable to cross the skin barrier naturally. Comprehensive skin care, therefore, must also address penetration of the components through the skin permeability barrier.

### SUMMARY OF THE INVENTION

The present invention provides compositions for the repair of mammalian skin. The compositions contain cell growth enhancers to increase the growth rate of skin cells, stimulators of cell growth enhancers, nutrients to support log phase growth of skin cells, cell protectors to protect growing cells and enhanced cellular activity, antioxidants to protect rejuvenated cells, extracellular matrix proteins, stimulators of extracellular matrix proteins, and penetration enhancers. The compositions of the present invention are effective for repairing and rejuvenating mammalian skin, such that aging skin treated with the compositions has a significant reduction in the number of fine lines and wrinkles in the skin. The compositions are also effective for promoting the healing of skin that has suffered a wound, such as a sunburn, cut, scrape, or abrasion.

In a first aspect, the present invention provides a composition for skin repair. In one embodiment, the composition contains 1) at least one cell growth enhancer and at least one stimulator of cell growth enhancers in an amount effective to increase the growth rate of skin cells; 2) a combination of nutrients in an amount effective to support log phase growth of skin cells; 3) at least one cell protector in an amount effective to protect growing cells and maintain enhanced cellular activity; 4) at least one antioxidant in an amount effective to protect rejuvenated cells from damage; 5) one or more extra-cellular matrix proteins in an amount effective to improve skin structure; 6) one or more stimulators of extra-cellular matrix protein production in an amount effective to increase the production of extra-cellular matrix proteins in the skin; 7) a mixture of penetration enhancers in an amount effective to allow penetration of the dermal layer by the cell growth enhancers, stimulators of cell growth enhancers, nutrients, cell protectors, antioxidants, extracellular matrix proteins, and stimulators of extracellular matrix protein production in an amount effective to promote skin repair; and 8) a biologically acceptable carrier.

The term “skin repair” refers to skin rejuvenation, the healing of a wound, or the moisturization of skin. The term “skin rejuvenation” or “rejuvenation of skin” refers to the prevention or reduction of fine lines and wrinkles in the skin, and that fine lines and wrinkles are reduced by 10% or more in number. The number of fine lines and wrinkles is calculated according to methods known in the art, such as using D-SQUAME® tape and image analysis (e.g., as in Example 11). “Corneocytes” are the dead keratin-filled squamous cells of the stratum corneum. “Fine lines” are the lines that appear in mammalian skin due to the effects of aging. Wrinkles are deeper than fine lines.

The term “wound healing” or “healing of a wound” as used herein refers to the repair of wounds due to a break in the skin barrier for example, by cuts, bruises, scrapes, sunburns, ulcer wounds, or wounds received during a surgical procedure or due to a burn. The wound may be, for example, caused by chemical burns from cosmetic facial peels or other dermatological and cosmetic surgery procedures. The term “cell growth enhancers” used herein means components that increase the rate of growth of the cells.

The term “skin cells” as used herein means cells found in human skin such as keratinocytes and fibroblasts. Rate of growth is determined by the amount of time necessary for a population of cells to double in number. Normal doubling times for young, healthy fibroblast cells are 24-30 hours. An amount of cell growth enhancers “effective to increase the growth rate of skin cells” is an amount effective to lower the doubling time of a population of fibroblasts that are doubling one time in more than every 35 hours from more than 35 hours to less than 32 hours. In another embodiment, the amount effective to increase the growth rate will decrease the doubling time of a population of fibroblasts by at least 10% or by at least 15% or by at least 20%. The term “stimulators of cell growth enhancers” used herein means components that further the increase of the rate of growth of the cells. The term “combination of nutrients” used herein means an amount of one or more nutritional ingredients in the compositions that can be effective in supporting log phase growth of skin cells such as human fibroblasts or keratinocytes.

Log phase growth is characterized by an exponential multiplication in the number of cells.

The term “cell protectors” as used herein means components that prevent cell death due to apoptosis, or that protect cell growth from the damaging effects of culture metabolites. Culture metabolites are compounds produced by cells when they grow and multiply. Some culture metabolites are harmful to cells and can result in slower cell growth or cell death, such as ammonia or urea. “Apoptosis” is defined as a normal series of events in a cell that leads to its death, also called programmed cell death. Compounds such as ammonia scavengers can be used for cell protection. Ammonia scavengers are combinations of certain amino acids (e.g., glutamic acid) in combination with vitamin B-6, and/or minerals that help remove ammonia from the blood. Other ammonia scavengers that bind with excess ammonia are L-arginine, creatine, and L-lysine. Cell death can be measured in-vitro by measuring the amount of lactate dehydrogenase in the cell culture medium. A lowering of lactate dehydrogenase indicates lower cell death in culture leading to improved cell growth. Standard laboratory assays are know that measure a reduction in the amount of lactate dehydrogenase in a cell culture medium. In one embodiment the prevention of cell death causes a 10% or greater reduction in lactate dehydrogenase in a standard cell culture.

The term “antioxidant” as used herein means components that prevents or impedes cell oxidation (destruction) by free radicals, etc thus providing protection to rejuvenated skin cells and maintaining log phase growth of cells. The term also includes components that provide protection from oxidation to components in the composition. An antioxidant is “effective to protect rejuvenated cells from damage” when it can maintain log phase growth of cells.

The term “extracellular matrix (ECM) proteins,” as used herein means proteins that provide the structure to the skin. Extracellular matrix proteins provide the cellular support structure or “scaffolding” underneath the epidermal layer of skin cells, and their degradation plays an important role in wrinkle formation of aging skin.

The term “stimulators of extracellular matrix protein production” as used herein means components that increase the production of extracellular matrix proteins by skin cells such as fibroblasts and keratinocytes. An ECM stimulator is “effective to increase the production of extracellular matrix proteins” when it increases the production of extracellular matrix proteins in fibroblasts or keratinocytes by 10% or more.

The term “penetration enhancers” as used herein means a compound that facilitates the movement of substances into and/or through the epidermis of the skin. The term “penetration enhancers” also includes the use of a transdermal delivery vehicle or a transdermal delivery device for enhancing penetration and delivery of the composition through the skin. The term “biologically acceptable carrier” as used herein, means a carrier suitable for topical application to mammalian skin.

In one embodiment the cell growth enhancer is one or more cell growth factors such as cytokines, regulatory factors, angiogenic factors, and adhesion proteins; the stimulator of cell growth enhancers is one or more of sodium hyaluronate, hyaluronic acid, ascorbic acid, sodium ascorbate, and amphiregulin; the mixture of nutrients effective to support log phase growth of skin cells can be one or more of carbohydrates, lipids, phospholipids, fatty acids, essential and non essential amino acids, inorganic salts, minerals, and trace metals, vitamins, nucleosides, purines, and pyrimidines, buffers, and other nutrients; the cell protector is insulin, transferrin, or selenium, and analogs and derivatives thereof; the antioxidant can be Vitamins E, Vitamin C, and Vitamin K, beta-carotene, coenzyme Q10, cysteine, glutathione, a thiol or analogs and derivatives thereof; the extra-cellular matrix protein can be fibrous proteins, and adhesion molecules; the stimulator of extra-cellular matrix protein production can be growth factors, chemical stimulators and adhesion proteins; and the penetration enhancer can be hydrophobic enhancers, hydrophilic enhancers, and hydrophobic and hydrophilic copolymers.

The term “cell growth factors” used herein means substances made by the body that function to regulate cell division and cell survival. “Growth factors” are naturally occurring proteins that cause cells to grow and divide. Cell growth factors include, but are not limited to, growth factors (e.g., epidermal growth factor (EGF), keratinocyte growth factor (KGF), fibroblast growth factor (acidic FGF), insulin-like growth factor (IGF), platelet derived growth factor (PDGF), granulocyte colony-stimulating factor (G-CSF), granulocyte-macrophage colony-stimulating factor (GM-CSF)), tissue growth factor (TGF-a), and other cell growth factors. “EGF” is a protein that stimulates epidermal cells to divide. “KGF” is a growth factor structurally related to fibroblast growth factor. “FGF” is a growth factor that has been isolated from a variety of cells. It has potent heparin-binding activity and is a potent inducer of DNA synthesis in a variety of normal diploid mammalian cell types from mesoderm and neuroectoderm lineages. It also has chemotactic and mitogenic activities. FGF has acidic and basic forms. “IGF” refers to insulin-like growth factors I and II. Insulin like growth factors I and II are polypeptides with sequence similarity to insulin. They are capable of eliciting the similar biological responses, including mitogenesis in cell culture. “PDGF” is an important mitogen that promotes growth in culture of cells of connective tissue origin. It consists of 2 different but homologous polypeptides A and B (˜30,000 D) linked by disulfide bonds, and plays a role in wound healing. “GCSF” is a glycoprotein containing internal disulfide bonds. It induces the survival, proliferation, and differentiation of neutrophilic granulocyte precursor cells and functionally activates mature blood neutrophils. “GMCSF” is an acidic glycoprotein with internal disulfide bonds. It stimulates the production of neutrophilic granulocytes, macrophages, and mixed granulocyte macrophage colonies from bone marrow cells and can stimulate the formation of eosinophil colonies from fetal liver progenitor cells. It also has some functional activities in mature granulocytes and macrophages. “TGF”, Transforming growth factor alpha, is a growth factor that stimulates growth of microvascular endothelial cells. Transforming growth factor beta stimulates wound healing. The transforming growth factor family includes many of the bone morphogenetic proteins. “Cytokines” are non-antibody proteins that act as intercellular mediators. They differ from classical hormones in that they are produced by a number of tissue cell types rather than by specialized glands. They generally act locally in a paracrine or autocrine rather than endocrine manner. Cytokines include, but are not limited to, hepatopoietin, erythropoietin and interleukins. “Interleukin” is the generic name for a group of well-characterized cytokines that are produced by leukocytes and other cell types. Examples of Interleukins include, but are not limited to, IL-1 through 18. “Regulatory factors” are proteins active in the activation or repression of transcription of the gene. Regulatory factors include, but are not limited to, human growth hormone and prostaglandin. “Angiogenic factors” are proteins that act to vascularize tissue and act in the development of new capillary blood vessels. Angiogenic factors include, but are not limited to, vascular endothelial growth factor (VEGF), fibroblast growth factor (acidic FGF), tissue growth factor (TGF-a), platelet derived growth factor (PDGF). “VEGF” is a protein that stimulates the growth of new blood vessels. “Adhesion proteins” are a large family of proteins that mediate direct contact between cells or between cells and the extracellular matrix during such physiological processes as cell activation, migration, proliferation, and differentiation. Adhesion proteins include, but are not limited to, fibronectin, vitronectin, laminin, thrombospondin, and tenasin.

In various embodiments at least one stimulator of cell growth enhancers is selected from the group that include, but is not limited to, sodium hyaluronate, hyaluronic acid, ascorbic acid, sodium ascorbate, and amphiregulin. In various embodiments the compositions contain these constituents in their native forms, but can also contain stimulators of these constituents. For example, amphiregulin and tissue growth factor alpha (TGF-α) may be used to induce and stimulate EGF production in cells.

“Carbohydrates” are the class of aldehyde or ketone derivatives of polyhydric alcohols, usually having hydrogen and oxygen in the proportion to form water, Cn(H₂O)ₙ. Carbohydrates include, but are not limited to, monosaccharides, disaccharides, polysaccharides and other sugars, derivatives and analogs thereof. “Lipids” are fatty substances present in blood and body tissues, including cholesterol and triglycerides. “Phospholipids” are esters of glycerol containing two fatty acids and a phosphate group, having nonpolar tails and polar heads, they tend to form bilayers in aqueous solution. “Fatty acids” are organic, monobasic acids derived from hydrocarbons by the equivalent of oxidation of a methyl group to an alcohol, aldehyde, and then acid. Fatty acids are saturated and unsaturated. Lipids, phospholipids, and fatty acids include, but are not limited to, linoleic acid, lipoic acid, cholesterol, and oleic acid, derivatives and analogs thereof. “Amino acids” are organic compounds that generally contain an amino (—NH₂) and a carboxyl (—COOH) group and are subunits that polymerize to form proteins. Essential and non-essential amino acids includes, but is not limited to, Alanine, Arginine.HCl, Asparagine, Aspartic Acid, Cystine.HCl, Cysteine.2HCl, Glutamic Acid, Glutamine, Glycine, Histidine.HCl, Isoleucine, Leucine, Lysine.HCl, Methionine, Phenylalanine, Proline, Serine, Threonine, Tryptophan, Tyrosine, Valine, (D and/or L forms) and their derivatives and analogs thereof. “Salts” are the neutral compounds formed by the union of an acid base. “Minerals” any naturally occurring substance that is neither vegetable nor animal. These are the most basic form into which organic matter can be broken. At the mineral stage, the particles are inorganic. “Trace Metals” are metals such as silver, copper, lead, cadmium, zinc, and mercury that normally occur in water and sediments at concentrations less than one part per million (ppm). Inorganic salts, minerals, and trace metals include, but are not limited to, copper sulfate, ferric nitrate, ferrous sulfate, potassium chloride, sodium chloride, sodium phosphate monobasic and dibasic, zinc sulfate, sodium acetate (anhydrous), sodium pyruvate, manganese sulfate, ammonium molybedate, nickel chloride, tin chloride, potassium phosphate monobasic and dibasic, sodium silicate, sodium selenite, and derivatives thereof. “Vitamins” are essential organic compounds required in trace amounts for normal growth and metabolic processes. They usually serve as components of coenzyme systems. Vitamins include but are not limited to, D-Biotin, Choline Chloride, Folic Acid, myo-Inositol, Niacinamide, D-Pantothenic Acid, calcium salt, Pyridoxal.HCl, Pyridoxine.HCl, Riboflavin, Thiamine.HCl, Vitamin B-12 and derivatives and analogs thereof. “Nucleosides and Nucleotides” are complex compounds of high molecular weight occurring in living cells. These are basically of two types, ribonucleic (RNA) and deoxyribonucleic (DNA) acids, both of which consist of nucleotides (nucleoside phosphates linked together by phosphate bridges). Nucleosides and nucleotides include, but are not limited to, Thymidine and derivatives thereof. Purines, and pyrimidines, but are not limited to, Hypoxanthine, and Adenine.HCl. “Purines” are a series of heterocyclic compounds that are variously substituted in nature and are known also as purine bases. They include adenine and guanine. “Pyrimidines” are a family of 6-membered heterocyclic compounds. They are planar and aromatic in character and include several nucleic acid constituents (cytosine, thymine, and uracil). “Buffers” are chemical systems that prevent change in the concentration of another chemical substance, e.g. proton donor and acceptor systems serve as buffers preventing marked changes in hydrogen ion concentration (pH). Buffers include, but are not limited to, HEPES, and sodium bicarbonate. Other nutrients include, but are not limited to, Putrescine.HCl, Vitamin A and D protein or protein extract, peptide, complex sources of carbon and nitrogen such as peptone and egg yolk, deoxyribose, ribose, serum or serum substitute, natural extract, plant or animal derived component.

The compositions may also contain components extracted from prokaryotic and eucaryotic cultures, such as proteins, lipoproteins or glycosylated protein fractions. In some embodiments nutrients include the components above in a modified form, for example modified to give hydrophilic, hydrophobic and/or lipophilic properties to enhance penetration into the skin. For example, glucose can be modified to octyl glucose, which enhances its delivery to the inner layers of the skin. Likewise, other modified components such as cetyl ascorbate or cetyl phosphoryl ascorbate can be used instead of (or in addition to) ascorbate. Also, lipophilic analogs of amino acids may be used. “Peptides” are any member of a class of compounds, which yield two or more amino acids on hydrolysis. They are formed by loss of water from the NH2 and COOH groups of adjacent amino acids. Peptides form the constituent parts of proteins. “Peptones” are any of the various products produced as a result of partial hydrolysis of proteins. “Natural extracts” are extracts are usually concentrated products obtained by treating a natural raw material with a solvent. The term “analog” used herein means a compound that resembles another in structure but is not necessarily an isomer (e.g., 5-fluorouracil is an analog of thymine); analog's are often used to block enzymatic reactions by combining with enzymes (e.g., isopropyl thiogalactoside vs. Lactose). The term “isomer” used herein means one of two or more molecules that have the same chemical formula but have a different stereochemical arrangement of their atoms. The term “derivative” used herein means a chemical substance derived from another substance either directly or by modification or partial substitution.

In various embodiments at least one cell protector is selected from the group that include, but are not limited to, insulin, transferrin, selenium, and derivatives thereof.

In various embodiments at least one antioxidant is selected from the group that include, but are not limited to, vitamins, beta-carotene, coenzyme Q10, cysteine, glutathione, a thiol or analogs and derivatives thereof. Vitamins include, but are not limited to, Vitamins E, Vitamin C, and Vitamin K. “Cysteine” is a sulfur containing amino acid found in many proteins “Glutathione” is the tripeptide glutamylcysteinylglycine. “Thiols” are similar to alcohol and phenol, except that the oxygen is replaced by sulfur (R—SH or Ar—SH).

In various embodiments at least one the extra-cellular matrix (ECM) protein is selected from each of the categories consisting of: fibrous proteins, and adhesion molecules. “Fibrous proteins” are insoluble proteins, including the collagens, elastins, fibrion, and keratins that are involved in structural or fibrous tissues. “Collagen” is the protein substance of the white fibers (collagenous fibers) of skin, tendon, bone, cartilage, and all other connective tissue, composed of molecules of tropocollagen, it is converted into gelatin by boiling. “Elastin” is a glycoprotein that is randomly coiled and cross-linked to form elastic fibers that are found in connective tissue. Like collagen, elastin composition is high in proline content. “Fibroin” is a variety of gelatin; the chief ingredient of raw silk, extracted as a white amorphous mass. “Keratins” are a fibrous scleroprotein that occurs in the outer layer of the skin and in horny tissues such as hair feathers nails and hooves. “Adhesion molecules” are a large family of proteins that mediate direct contact between cells or between cells and the extracellular matrix during such physiological processes as cell activation, migration, proliferation, and differentiation. Examples of adhesion molecules include, but are not limited to, Cadherins, Immunoglobulin superfamily, Integrins, Proteoglycans, and Selectins. “Cadherins” are a group of functionally related glycoproteins responsible for the calcium-dependent cell-to-cell adhesion mechanism. They are divided into subclasses E-, P-, and N-cadherins, which are distinct in immunological specificity and tissue distribution. They promote cell adhesion via a homophilic mechanism. These compounds play a role in the construction of tissues and of the whole animal body. “Immunoglobulin superfamily” are a class of proteins produced in lymph tissue in vertebrates and that function as antibodies in the immune response. “Integrins” are a superfamily of cell surface proteins. Most integrins are heterodimeric with a subunit of about 95 kD that is conserved through the superfamily and a more variable subunit of 150-170 kD. They interact with a wide variety of ligands including extracellular matrix glycoproteins, complement, and other cells, while their intracellular domains interact with the cytoskeleton. The integrins consist of at least three identified families: the cytoadhesin receptors, the leukocyte adhesion receptors, and the very-late-antigen receptors. Each family contains a common beta-subunit combined with one or more distinct alpha-subunits. These receptors participate in cell-matrix and cell-cell adhesion in many physiologically important processes, including embryological development, hemostasis, thrombosis, wound healing, immune and nonimmune defense mechanisms, and oncogenic transformation. “Glycoproteins” are conjugated protein-carbohydrate compounds including mucins, mucoid, and amyloid glycoproteins. A “Proteoglycan” is a complex molecule containing at least one glucosaminoglycan bound to a core protein. Proteoglycans include, but are not limited to, versican, decorin, betaglycan, syndecan and aggrecan. “Glucosaminoglycans” are heteropolysaccharides, which contain an N-acetylated hexosamine in a characteristic repeating disaccharide unit. The repeating structure of each disaccharide involves alternate 1,4- and 1,3-linkages consisting of either N-acetylglucosamine or N-acetylgalactosamine. Glucosamineglycans include, but are not limited to, chondroitin sulfate, dermatan sulfate, heparin sulfate, heparin, keratin sulfate and hyaluronan. “Selectins” are lectin-like adhesion molecules that are important in the intitial interactions between leukocytes and endothelial cells during the margination process. Compositions of the invention use components in native forms or analogs thereof.

In various embodiments at least one stimulator of extra-cellular matrix protein production is selected from each of the categories consisting of: 1) growth factors, 2) chemical stimulators, and 3) adhesion proteins. Growth factors include, but are not limited to, transforming growth factor-β, (TGF-β) and isoforms like β1, β2, β3, and fibroblast growth factor (basic FGF), vascular endothelial growth factor (VEGF). Chemical stimulators include, but are not limited to, sodium ascorbate, hyaluronic acid, tretinoin (RETIN-A®) and analogs, heparin-sodium salt and analogs thereof. Cell adhesion proteins include, but are not limited to, hyaluronic acid, fibronectin, vitronectin, laminin, thrombospondin, tenasin, stress proteins such as GR78, and short chain peptides.

In various embodiments at least one stimulators of penetration enhancement is selected from a each of the groups consisting of 1) hydrophobic enhancers, 2) hydrophilic enhancers, and 3) hydrophobic and hydrophilic block copolymers. “Hydrophobic enhancers” are compounds that tend to be electrically neutral and nonpolar, and thus preferring other neutral and nonpolar solvents or molecular environments. Hydrophobic enhancers include, but are not limited to, lipids such as oils including mineral oil, essential oil, herbal oil, animal oil, synthetic oil, natural oil, camphor, menthane, terpenes and lipoproteins. “Mineral oil” is any oil made from mineral sources, e.g., petroleum. “Essential oils” are plant products, usually somewhat volatile, giving the odors and tastes characteristic of the particular plant, thus possessing the essence, e.g., citral, pinene, almond oil, coconut oil, linseed oil, camphor, menthane, terpenes, usually, the steam distillate or plants or oils of plants obtained by pressing out the rinds of a particular plant. “Lipoproteins” are particles composed of proteins and lipids (triglycerides, phospholipids and cholesterol) that enable lipids (which are water insoluble) to be carried in blood plasma. Examples of lipoprotein include, but are not limited to, shea butter, cacao butter, triglycerides, plant lipoproteins such as wheat, soy, animal lipoproteins such as milk. “Hydrophilic enhancers” are compounds that tend to be typically electrically polarized and capable of H-bonding, enabling them to dissolve more readily in water than in oil or other “non-polar” solvents. Hydrophilic enhancers include, but are not limited to, fatty acids and fatty alcohols, detergents, alcohols, and glycols. “Fatty acids” are a large group of monobasic acids, especially those found in animal and vegetable fats and oils, having the general formula CₙH₂n+₁COOH. Characteristically made up of saturated or unsaturated aliphatic compounds with an even number of carbon atoms, this group of acids includes, but is not limited to, palmitic, stearic, and oleic acids. A “fatty alcohol” is an alcohol with long, unbranched carbon chains, derived mainly from petrochemical products. Fatty alcohols include, but are not limited to, cetyl alcohol (natural and iso-), oleyl alcohol, cetyl-stearyl alcohol, octyl alcohol, decyl alcohol, ricinol alcohol, lauryl alcohol, strearyl alcohol, lauro-myristyl alcohol, tallow alcohol. “Detergents” include, but are not limited to, ionic and nonionic surfactants, polysorbate 80, polyoxyethylenesorbitan monooleate (TWEEN® 80), TRITON™ X100 surfactant, sodium dodecylsulfate (SDS) and sulfated higher alcohols or derivatives thereof. “Alcohols” include, but are not limited to, methanol, ethanol, propanol, isopropanol, glycols and analogs and derivatives thereof. “Glycols” include, but are not limited to, short chain glycols such as propylene glycol and butylene glycol and analogs. “Hydrophobic and Hydrophilic copolymers” tend to combine the properties of Hydrophobic and Hydrophillic enhancers. Hydrophobic and hydrophilic block copolymers include, but are not limited to, proprietary compounds such as PLURONIC® F68. Components may be a linked to a peptide, etc. to improve transport through the skin. Likewise, VEGF may be linked to a detergent molecule such as polyoxyethylenesorbitan monooleate (TWEEN® 80) for enhanced delivery. The term “penetration enhancers” also includes those compounds described above chemically modified to impart hydrophilic, hydrophobic and/or lipophilic properties to the compounds. The term also includes derivative, analogs, isoforms, precursors and subunits of the compounds described above.

In various embodiments, penetration enhancement of the composition may include a transdermal delivery vehicle, or a transdermal delivery device to enhance penetration of components. The term “delivery vehicle” as used herein means a mechanism for delivering the compositions to the skin cells. Examples of a transdermal delivery vehicle include, but are not limited to, liposomes, micelles, emulsions and micro-emulsions such oil-in-water, water-in-oil, water-in-oil-in-water, and oil-in-water-in-silicone emulsions, aqueous solutions such as hydro-alcoholic solvent systems and non-aqueous solutions such as oil, ethanol, isopropanol, dimethicone, cyclomethicone, dimethlysulfoxide, solids, powders, gels or films, serum, cream, or silicones to carry composition ingredients into the skin. The term “delivery device” as used herein means devices that are used to enhance penetration and delivery of the compositions to the skin cells. Examples of transdermal delivery device include, but are not limited to, electrical devices such as ionotophoresis, pumps, a transdermal patch, an occlusive pad, mask or dressing, adhesive or non-adhesive bandage, a gel or film.

In various embodiments the compositions are provided in a biologically acceptable carrier for topical application. Thus, a biologically acceptable carrier can be applied to mammalian skin without causing undue toxicity, irritation, allergic response, and the like. The formulations of the invention may be prepared in combination with additional ingredients such as sunscreens, moisturizers, exfoliators, cosmeceuticals, and the addition of appropriate carriers and bulking agents such as gums, resins, waxes, polymers, salts, and the like. Formulations may be composed in the form of sprays using either mechanical pump containers or pressurized aerosol containers using conventional propellants. The term “topical application”, as used herein, means any means of application to the surface of the skin. “Moisturization” refers to a 5% or greater increase in the moisture level of the skin, as measured with a NOVA™ DPM (dermal phase meter) meter or equivalent (Nova Technologies). In other embodiments, moisturization refers to a 10% or greater or 15% or greater or 20% or greater increase in the moisture level of the skin, using the same measurement.

In another aspect the present invention provides methods for repairing mammalian skin. The methods include contacting the skin with a composition of the invention, allowing the composition to remain in contact with the skin for a period of time sufficient for the cell growth enhancers and stimulators of cell growth enhancers, nutrients, cell protectors, antioxidants, extracellular matrix proteins, and stimulators of extracellular matrix protein production to permeate mammalian skin in amounts effective to repair the skin, and thereby repair the mammalian skin. In preferred embodiments of the invention, the mammal is a human. In other embodiments, the compositions are applied as a coating on medical or surgical devices, such as sutures, implants, homeostatic plugs, dressings, gauze and pads. A “biologically effective amount” is an amount effective to perform the function that is described for the individual component. In various embodiments the compositions can be applied through repeated applications, such as each evening at bedtime, or daily. The compositions can remain on the skin for a convenient period of time, for example, 6 hours, 8 hours, 10 hours, 12 hours, or any period of time the user deems convenient.

In another aspect the present invention provides methods for increasing hair growth on the scalp. The methods include contacting the skin of the scalp with a composition of the invention, allowing the composition to remain in contact with the skin for a period of time sufficient for the cell growth enhancers, nutrients, extracellular matrix proteins, and stimulators of extracellular matrix to permeate mammalian skin in amounts effective to increase hair growth, thereby increasing the growth of hair on the scalp. An increase in the growth of hair on the scalp means a 5% or greater increase in weight of hair clippings or hair count on the treated scalp area. In various embodiments, the clippings can be taken after 3 months or 6 months of treatment, or any period of time sufficient to provide a statistically meaningful result. In preferred embodiments, persons with healthy hair on the scalp can realize an increase in hair growth. The present methods are also useful for preventing hair loss. “Preventing hair loss” means that the loss of hair from the scalp is slowed.

The summary of the invention described above is not limiting and other features and advantages of the invention will be apparent from the following detailed description of the preferred embodiments, as well as from the claims.

### DETAILED DESCRIPTION OF THE INVENTION

The present invention relates to the field of topical skin care, cosmetics, cosmeceuticals, skin rejuvenation, hair care, skin anti-aging, skin repair, and wound healing. The invention directly addresses the more important and complex causes of skin aging caused by the age-induced slowing of the division rate of the skin cells and the defective cross-linking of collagen and elastin fibers. The compositions described herein provide skin care compositions that rejuvenate and protect skin cells at both the epidermal and the dermal layers, stimulate skin cells to increase extracellular skin matrix production, and enhance the penetration of composition ingredients through the skin permeability barrier.

The compositions of the present invention are formulated for comprehensive skin rejuvenation, protection and skin repair, and contain a unique combination of components provided in a biologically acceptable carrier. In preferred embodiments the compositions are topically applied to the surface of treated skin. The composition ingredients form the building blocks for complete care of skin cells from a scientific and biological standpoint.

Cell growth enhancers provided in the compositions increase the growth rate of skin cells to approach the rate of growth observed in the skin of persons below 20 years of age. Cell protectors and antioxidants protect rejuvenated cells. Nutrients and nutritional factors provided in the composition provide nourishment and energy to maintain cells in the rejuvenated mode. Extracellular matrix proteins and stimulators of extracellular matrix protein production serve to replenish, produce and maintain matrix and structure of the skin. Penetration enhancers ensure that components penetrate the skin barrier and are delivered to the skin cells. The compositions are formulated to effective concentrations so as to provide rejuvenating effects on the skin such that normal, youthful, skin function is achieved and maintained. Since these compositions contain the essential building blocks for skin protection repair, they find wide applications in skin repair as in treating sunburns, radiation-bums, scrapes, superficial bums, or for use in cosmetic surgery procedures such as facial peels. The compositions also find application in wound healing such as repair of cuts, bums, ulcer and incision wounds, and can also be applied as a coating on medical or surgical instruments such as threads for sutures, prosthetic implants, homeostatic plugs, and wound dressings.

In a highly preferred embodiments the compositions may be used to prevent or reverse the physical effects of skin aging. Examples include, but are not limited to, topical applications of the compositions to protect and maintain youthful skin texture and prevent fine lines and wrinkles from forming as a person ages. In a preferred embodiment the compositions may be used in the rejuvenation of aged skin. For example, the compositions can be topically applied to improve skin texture and reduce the number of fine lines and wrinkles. In another embodiment the compositions can contain varying concentrations of the constituents in the treatment of different skin conditions. For example, one embodiment can contain a higher concentration of enhancers and stimulators to increase cell growth. In another embodiment the composition can contain higher concentrations of cell protectors and antioxidants to shield rejuvenated, growing cells from damage and maintain enhanced cellular activity. In another embodiment the composition can contain a higher concentration of nutrients for maintaining reactivated cells. In yet another embodiment the compositions can contain a higher concentration of extracellular matrix and stimulators of extracellular matrix protein production to reduce the number of lines in heavily wrinkled skin. A formulation containing balanced concentrations in the composition may be used to maintain rejuvenated, youthful skin.

In another embodiment the compositions may be used for the care of skin of the scalp that bears hair. For example, the compositions are topically applied to the scalp to prevent hair loss or increase hair growth from the follicles.

In another embodiment the compositions may be applied for the repair of damaged skin. Damaged skin is skin that has suffered a trauma, such as an abrasion, sunburns, scrapes, superficial bums. The compositions can also be used to aid the healing of skin after cosmetic and reconstructive surgery procedures, or after a cosmetic facial peel procedure. Other examples of healing damaged skin include, but are not limited to applications to promote healing of wounds such as cuts, burns, ulcer and incision wounds. The compositions of the invention are topically applied to the skin to be treated. The compositions can be administered as a film, mask, spray, or ointment. In another embodiment the compositions are used as a coating of medical or surgical devices, such as sutures, implants, homeostatic plugs, dressings, gauze and pads.

The compositions may also be formulated as suspensions for improved penetration and delivery. For example the composition or its components may be contained in liposomes, micelles, emulsions and micro-emulsions. Oil-in-water, water-in-oil, water-in-oil-in-water, and oil-in-water-in-silicone emulsions are useful in the present invention. Other examples of delivery vehicles include, but are not limited to aqueous solutions such as hydro-alcoholic solvent systems and non-aqueous solutions such as oil, ethanol, isopropanol, dimethicone, cyclomethicone, dimethlysulfoxide, solids, powders, gels or films, serum, cream, or silicones. The composition may use a transdermal delivery device for enhanced penetration of its components. Examples of delivery devices include, but are not limited to, electrical devices such as ionotophoresis, pumps, a transdermal patch, an occlusive pad, mask or dressing, adhesive or non-adhesive bandage, a gel or film.

### Preparations and Applications

The compositions of the present invention are prepared by mixing of the ingredients in one or more steps, with or without heating or cooling. Moisturizers, absorption enhancers, sunscreens, color, preservatives, and fragrance may also be added to the formulations as desired.

The compositions of the invention are useful for rejuvenating and protecting skin cells to prevent, and/or slow down skin aging effects, improve skin texture and prevent and reduce fine lines and wrinkles, and rejuvenate hair follicles and prevent hair loss. The compositions are for general cosmetic use and for specific skin treatments. For example a treatment regimen may use formulations of nutrients, cell growth enhancers, and stimulators of extra-cellular matrix production to rejuvenate aging skin. A composition rich in nutrients may be used for a defined period to energize the cells and prepare them for application of the complete composition. A composition especially rich in cell growth enhancers may be used to improve cell growth in aging skin. A composition rich in extracellular matrix stimulators may be used to improve skin structure. In preferred embodiments, formulations containing a balanced mixture of the ingredients mentioned above may be used to achieve and maintain healthy, rejuvenated skin. The compositions of the invention are also useful for the prevention of hair loss from the skin of the scalp, or to decrease the rate of hair loss from the scalp.

The compositions of the invention are useful for repairing damaged skin. For example, topical applications may be used to promote healing of damaged skin. Other topical applications include healing of facial peels and other cosmetics surgery procedures, healing of non-diseased wounds such as cuts, bums, ulcer and incision wounds, or as a wound healing promoter such as a coating of medical or surgical device essentially consisting of sutures, implants, homeostatic plugs, dressings, gauze and pads. In any or all of these applications the composition may be administered as a gel, film or mask, spray or ointment.

The invention is further illustrated in the following examples. These examples are given solely for the purpose of illustration and are not to be construed as limitations of the present invention as many variations thereof are possible without departing from the spirit and scope of the invention.

The following are exemplary compositions in accordance with this invention. Many variations of compositions, components and ingredients (selected from groups stated in description of terms) can be effectively employed for the applications of the invention. Amounts are given as examples only and higher and lower amounts can also be effectively employed. Formulations may contain commonly used cosmetic ingredients such as sunscreens, moisturizers, exfoliators, active cosmeceuticals, preservatives, antibacterial agents, anti-fungal agents, antiviral agents, antibiotics, color, fragrance and appropriate carriers and bulking agents such as gums, resins, waxes, polymers, salts, and the like.

### EXAMPLE 1

This example provides an example of the active components of a composition for rejuvenating skin. This composition rejuvenates the skin and improves the texture of the skin. The person of ordinary skill will realize that many variations of this preparation can be made by varying the ingredients to address particular needs of a situation. The composition is prepared as an emulsion with other commonly used cosmetic ingredients.

| COMPONENTS | INGREDIENT | Conc. Range (mg/L) |
|---|---|---|
| Cell growth enhancers | | |
| Growth factor | EGF | 0.0001-10.0 |
| Other factors | VEGF | 0.00001-10.0 |
| Stimulators of growth enhancers | hyaluronic acid | 10.0-10,000.0 |
| | sodium ascorbate | 10-1,000.0 |
| Nutrients | | |
| carbohydrates | D-Glucose | 100-10,000.0 |
| lipids, phospholipids, fatty acids | selected | 0.001-1,000.0 |
| amino acids | selected | 0.04-4,000.0 |
| salts, minerals, trace metals | selected | 0.00001-10,000.0 |
| vitamins | selected | 0.0001-1,000.0 |
| nucleosides | selected | 0.001-100.0 |
| purines, and pyrimidines | hypoxanthine | 0.01-1,000.0 |
| buffers | HEPES | 100.0-10,000.0 |
| others | putrescine-HCl | 0.001-10000.0 |
| Cell Protectors | insulin | 0.1-100.0 |
| | transferrin | 0.1-100.0 |
| | selenium | 0.1-100.0 |
| Antioxidants | vitamin E | 0.05-5.0% |
| | vitamin C | 0.01-4.0% |
| Extra Cellular Matrix | | |
| fibrous protein | collagen | 0.1-3.0% |
| adhesion molecules | fibronectin | 0.00001-10.0 |
| Stimulators of ECM | | |
| growth factors | TGF-b | 0.00001-10.0 |
| | basic FGF | 0.00001-10.0 |
| chemical stimulators | sodium L- | 10-1,000.0 |
| | ascorbate | |
| | hyaluronic acid | 10.0-10,000.0 |
| adhesion proteins | fibronectin | 0.00001-10.0 |
| Penetration Enhancers | | |
| hydrophobic enhancers | mineral oil | 0.1-50.0% |
| | essential oils | 0.1-50.0% |
| hydrophilic enhancers | Polysorbate 80 | 0.0001-1.0% |
| | cetyl alcohol | 10-1,000.0 |
| hydrophobic and hydrophilic | PLURONIC ® | 10-1,000.0 |
| copolymers | F68 | |

### EXAMPLE 2

This example provides an example of the active components of a composition suited for skin repair, such as the healing of superficial burns caused by facial peels. The formulation is prepared as a cream with other commonly used cosmetic ingredients.

| COMPONENTS | INGREDIENT | Conc. Range (mg/L) |
|---|---|---|
| Cell growth enhancers | | |
| Growth factor | EGF | 0.0001-10.0 |
| Other factors | VEGF | 0.00001-10.0 |
| Stimulators of | sodium hyaluronate | 10.0-10,000.0 |
| growth enhancers | sodium ascorbate | 10-1,000.0 |
| Nutrients | | |
| carbohydrates | D-Glucose | 100-10,000.0 |
| lipids, phospholipids, | selected | 0.001-1,000.0 |
| fatty acids | | |
| amino acids | selected | 0.04-4,000.0 |
| salts, minerals, trace metals | selected | 0.00001-10,000.0 |
| vitamins | selected | 0.0001-1,000.0 |
| nucleosides | selected | 0.001-100.0 |
| purines, and pyrimidines | hypoxanthine | 0.01-1,000.0 |
| buffers | HEPES | 100.0-10,000.0 |
| others | putrescine-HCl | 0.001-10000.0 |
| Cell Protectors | insulin | 0.1-100.0 |
| | transferrin | 0.1-100.0 |
| | selenium | 0.1-100.0 |
| Antioxidants | vitamin E | 0.05-5.0% |
| | vitamin C | 0.01-4.0% |
| Extra Cellular Matrix | | |
| fibrous protein | collagen | 0.1-3.0% |
| adhesion molecules | fibronectin | 0.00001-10.0 |
| Stimulators of ECM | | |
| growth factors | TGF-b | 0.00001-10.0 |
| | basic FGF | 0.00001-10.0 |
| chemical stimulators | sodium L-ascorbate | 10-1,000.0 |
| | hyaluronic acid | 10.0-10,000.0 |
| adhesion proteins | fibronectin | 0.00001-10.0 |
| Penetration Enhancers | | |
| hydrophobic enhancers | mineral oil | 0.1-50.0% |
| hydrophilic enhancers | lipoic acid | 0.001-1,000.0 |
| hydrophobic and | SYNPERONIC ® F68 | 10-1,000.0 |
| hydrophilic copolymers | | |

### EXAMPLE 3

This example provides the active components of a composition for wound healing, such as cuts, surgical incisions, or other breaks in the skin. The formulation is prepared as an ointment with an antimicrobial agent or antibiotics and other commonly used cosmetic ingredients.

| COMPONENTS | INGREDIENT | Conc. Range (mg/L) |
|---|---|---|
| Cell growth enhancers | | |
| Growth factor | EGF | 0.0001-10.0 |
| | acidic FGF | 0.0001-10.0 |
| Other factors | VEGF | 0.00001-10.0 |
| | fibronectin | 0.00001-10.0 |
| Stimulators of | hyaluronic acid | 10.0-10,000.0 |
| growth enhancers | ascorbic acid | 10-1,000.0 |
| Nutrients | | |
| carbohydrates | D-Glucose | 100-10,000.0 |
| lipids, phospholipids, | selected | 0.001-1,000.0 |
| fatty acids | | |
| amino acids | selected | 0.04-4,000.0 |
| salts, minerals, trace metals | selected | 0.00001-10,000.0 |
| vitamins | selected | 0.0001-1,000.0 |
| nucleosides | selected | 0.001-100.0 |
| purines, and pyrimidines | hypoxanthine | 0.01-1,000.0 |
| buffers | HEPES | 100.0-10,000.0 |
| others | putrescine-HCl | 0.001-10000.0 |
| Cell Protectors | insulin | 0.1-100.0 |
| | transferrin | 0.1-100.0 |
| | selenium | 0.1-100.0 |
| Antioxidants | vitamin E | 0.05-5.0% |
| | vitamin C | 0.01-4.0% |
| | vitamins K | 0.05-5.0% |
| Extra Cellular Matrix | | |
| fibrous protein | collagen | 0.1-3.0% |
| adhesion molecules | vitronectin | 0.00001-10.0 |
| | glucosamineglycans | 0.00001-10.0 |
| Stimulators of ECM | | |
| growth factors | TGF-b | 0.00001-10.0 |
| | basic FGF | 0.00001-10.0 |
| chemical stimulators | sodium L-ascorbate | 10-1,000.0 |
| | hyaluronic acid | 10.0-10,000.0 |
| adhesion proteins | vitronectin | 0.00001-10.0 |
| Penetration Enhancers | a transdermal | |
| | delivery vehicle | |
| | or device | |

### EXAMPLE 4

This example provides the active components of a composition to promote hair growth on the scalp or to decrease the rate of hair loss on the scalp. The formulation is prepared as an oil, with preservatives, and other commonly used cosmetic ingredients.

| COMPONENTS | INGREDIENT | Conc. Range (mg/L) |
|---|---|---|
| Cell growth enhancers | | |
| Growth factor | EGF | 0.0001-10.0 |
| | acidic FGF | 0.0001-10.0 |
| Other factors | VEGF | 0.00001-10.0 |
| | fibronectin | 0.00001-10.0 |
| Stimulators of growth | sodium hyaluronate | 10.0-10,000.0 |
| enhancers | ascorbic acid | 10-1,000.0 |
| Nutrients | | |
| carbohydrates | D-Glucose | 100-10,000.0 |
| lipids, phospholipids, | selected | 0.001-1,000.0 |
| fatty acids | | |
| amino acids | selected | 0.04-4,000.0 |
| salts, minerals, trace metals | selected | 0.00001-10,000.0 |
| vitamins | selected | 0.0001-1,000.0 |
| nucleosides | selected | 0.001-100.0 |
| purines, and pyrimidines | hypoxanthine | 0.01-1,000.0 |
| buffers | HEPES | 100.0-10,000.0 |
| others | putrescine-HCl | 0.001-10000.0 |
| Cell Protectors | insulin | 0.1-100.0 |
| | transferrin | 0.1-100.0 |
| | selenium | 0.1-100.0 |
| Antioxidants | vitamin E | 0.05-5.0% |
| | vitamin C | 0.01-4.0% |
| | vitamins K | 0.05-5.0% |
| Extra Cellular Matrix | | |
| fibrous protein | collagen | 0.1-3.0% |
| | α-keratins | 0.1-3.0% |
| | elastins | 0.1-3.0% |
| adhesion molecules | fibronectin | 0.00001-10.0 |
| Stimulators of ECM | | |
| growth factors | TGF-b | 0.00001-10.0 |
| | basic FGF | 0.00001-10.0 |
| chemical stimulators | sodium L-ascorbate | 10-1,000.0 |
| | hyaluronic acid | 10.0-10,000.0 |
| adhesion proteins | glucosamineglycans | 0.00001-10.0 |
| Penetration Enhancers | a transdermal delivery | |
| | vehicle or device | |
| hydrophobic enhancers | mineral oil | 0.1-50.0% |
| | essential oils | 0.1-50.0% |
| hydrophilic enhancers | lipoic acid | 0.001-1,000.0 |
| | cetyl alcohol | 10-1,000.0 |
| hydrophobic and | PLURONIC ® F68 1 | 0-1,000.0 |
| hydrophilic block | | |
| copolymers | | |

### EXAMPLE 5

This example provides another illustration of the active components of a composition of the present invention for rejuvenating skin.

| COMPONENT | INGREDIENT | Conc. Range (mg/L) | Target Conc. (mg/L) | |
|---|---|---|---|---|
| Cell growth enhancers | | | | |
| Growth factors | TGF-a | 0.0001-10.0 | 0.1 | |
| Other factors | fibronectin | 0.00001-10.0 | 0.001 | |
| Stimulators of growth | hyaluronic acid | 10.0-10,000.0 | 50 | |
| enhancers | | | | |
| | ascorbic acid | 10-1,000.0 | 150 | |
| Nutrients | | | | |
| carbohydrates | D-Glucose | 100-10,000.0 | 4000 | |
| lipids, phospholipids, | selected | 0.001-1,000.0 | 0.001-1,000.0 | |
| fatty acids | | | | |
| amino acids | selected | 0.04-4,000.0 | 0.04-4,000.0 | |
| salts, minerals, trace | selected | 0.00001-10,000.0 | 0.00001-10,000.0 | |
| metals | | | | |
| vitamins | selected | 0.0001-1,000.0 | 0.0001-1,000.0 | |
| nucleosides | selected | 0.001-100.0 | 0.001-100.0 | |
| purines, and | hypoxanthine | 0.01-1,000.0 | 0.01-1,000.0 | |
| pyrimidines | | | | |
| buffers | HEPES | 100.0-10,000.0 | 6,000.0 | |
| others | putrescine-HCl | 0.001-10000.0 | 0.1 | |
| Cell Protectors | insulin | 0.1-100.0 | 5.0 | |
| | transferrin | 0.1-100.0 | 5.0 | |
| | selenium | 0.1-100.0 | 0.005 | |
| Antioxidants | vitamin E | 0.05-5.0% | 1,000.0 | |
| | vitamin C | 0.01-4.0% | 1,000.0 | |
| Extra Cellular Matrix | | | | |
| fibrous proteins | collagen | 0.1-3.0% | 2,000.0 | |
| adhesion molecules | fibronectin | 0.00001-10.0 | 0.001 | |
| Stimulators of ECM | | | | |
| growth factors | TGF-b | 0.00001-10.0 | 0.001 | |
| | basic FGF | 0.00001-10.0 | 0.001 | |
| chemical stimulators | sodium L-ascorbate | 10-1,000.0 | 150 | |
| | hyaluronic acid | 10.0-10,000.0 | 50 | |
| adhesion proteins | fibronectin | 0.00001-10.0 | 0.001 | |
| Penetration Enhancers | | | | |
| hydrophobic | mineral oil | 0.1-50.0% | 10 | ml/L |
| enhancers | | | | |
| hydrophilic enhancers | Polysorbate 80 | 0.0001-1.0% | 1.0 | ml/L |
| hydrophobic and | PLURONIC ® F68 | 10-1,000.0 | 250.0 | |
| hydrophilic block | | | | |
| copolymers | | | | |

### EXAMPLE 6

This example provides another illustration of the active components of a composition of the present invention for rejuvenating skin.

| COMPONENT | INGREDIENT | Conc. Range (mg/L) | Target Conc. (mg/L) | |
|---|---|---|---|---|
| Cell growth enhancers | | | | |
| Growth factors | TGF-a | 0.0001-10.0 | 0.01 | |
| Other factors | fibronectin | 0.00001-10.0 | 0.00005 | |
| Stimulators of growth | Sodium hyaluronate | 10.0-10,000.0 | 5.0 | |
| enhancers | | | | |
| | ascorbic acid | 10-1,000.0 | 150 | |
| Nutrients | | | | |
| carbohydrates | D-Glucose | 100-10,000.0 | 4000 | |
| lipids, phospholipids, | selected | 0.001-1,000.0 | 0.001-1,000.0 | |
| fatty acids | | | | |
| amino acids | selected | 0.04-4,000.0 | 0.04-4,000.0 | |
| salts, minerals, trace | selected | 0.00001-10,000.0 | 0.00001-10,000.0 | |
| metals | | | | |
| vitamins | selected | 0.0001-1,000.0 | 0.0001-1,000.0 | |
| nucleosides | selected | 0.001-100.0 | 0.001-100.0 | |
| purines, and | hypoxanthine | 0.01-1,000.0 | 0.01-1,000.0 | |
| pyrimidines | | | | |
| buffers | HEPES | 100.0-10,000.0 | 6,000.0 | |
| others | putrescine-HCl | 0.001-10000.0 | 0.1 | |
| Cell Protectors | insulin | 0.1-100.0 | 5.0 | |
| | transferrin | 0.1-100.0 | 5.0 | |
| | selenium | 0.1-100.0 | 0.005 | |
| Antioxidants | vitamin E | 0.05-5.0% | 1,000.0 | |
| | vitamin C | 0.01-4.0% | 1,000.0 | |
| Extra Cellular Matrix | | | | |
| fibrous proteins | collagen | 0.1-3.0% | 1,000.0 | |
| adhesion molecules | fibronectin | 0.00001-10.0 | 0.0001 | |
| Stimulators of ECM | | | | |
| growth factors | TGF-b | 0.00001-10.0 | 0.0001 | |
| | basic FGF | 0.00001-10.0 | 0.0001 | |
| chemical stimulators | sodium L-ascorbate | 10-1,000.0 | 150 | |
| | hyaluronic acid | 10.0-10,000.0 | 5.0 | |
| adhesion proteins | fibronectin | 0.00001-10.0 | 0.00005 | |
| Penetration Enhancers | | | | |
| hydrophobic | mineral oil | 0.1-50.0% | 10 | ml/L |
| enhancers | | | | |
| hydrophilic enhancers | Polysorbate 80 | 0.0001-1.0% | 1.0 | ml/L |
| hydrophobic and | PLURONIC ® F68 | 10-1,000.0 | 25.0 | |
| hydrophilic block | | | | |
| copolymers | | | | |

### EXAMPLE 7

This example describes an in vitro study demonstrating transport into and through the skin of a composition of the invention using percutaneous absorption in human cadaver skin. The model uses human cadaver skin mounted in specially designed diffusion chambers, which allow the skin to be maintained at a temperature and humidity corresponding to in vivo conditions.

Human cadaver trunk skin, without obvious signs of skin disease, is obtained within 24 hours of death from a skin bank. The skin is dermatomed to approximately 0.25 mm, sealed in a water-impermeable plastic bag and stored at −70° C. until the day of the experiment. Prior to use the skin is thawed by placing the bag in a 37° C. water bath and rinsing it in tap water to remove adherent blood or other materials from the surface. Skin from a single donor is cut into multiple smaller sections large enough to fit on 2.0 cm² Franz diffusion cells. The dermal chamber is filled to capacity with a receptor solution of PBS, pH 7.4, and the epidermal chamber is left open. The Franz cells are then placed in a diffusion apparatus in which the dermal receptor solution is stirred magnetically at 600 rpm and its temperature is maintained at 37° C.

At the start of the study, the receptor solution is replaced with a fresh solution of PBS. Another solvent may be used in place of PBS if the composition is not soluble in water, to improve recovery. Each test composition is applied to triplicate sections (tape stripped skin, if exfoliation conditions need to be simulated) of donor skin at a target dose of 2-10 ml/cm². The donor chimney is covered with a water impermeable barrier (e.g. plastic wrap) to prevent evaporation. At 24 and 48 hours after dosing, the receptor solution is removed and replaced with fresh solution. The harvested receptor solution is analyzed for the presence and concentration of composition components.

Total absorption of a component is measured by the total amount of material collected over 48 hours (in all receptor solutions) and a percent absorption is calculated from the amount applied. The rate of absorption of component material is calculated over the 48-hour period. The percent absorption and the rate of absorption of components are used to compare compositions for penetration characteristics. Detectable presence of composition components in the receptor solution demonstrates that the components of the compositions penetrate the skin barrier and will reach the inner layers of the skin, in vivo, when applied topically to the skin.

### EXAMPLE 8

This example describes an in vitro study for demonstrating sustained cell nourishment and an increase in the cell growth rate achieved with the use of the present invention. Concentrations of test composition permeating the skin are demonstrated, as determined from percutaneous absorption testing. Phosphate buffer saline (PBS) is used as the control.

Human dermal fibroblast cells (obtained from a culture collection such as ATCC) are inoculated at 0.5 million cells in T-150 flasks containing 40 ml of DMEM/F12 supplemented with 10% bovine calf serum and grown in a 5% CO₂ incubator at 37° C. Confluent T-flask culture (usually grown for 12 days) are trypsinized, and the cells counted and collected.

0.5 million human dermal fibroblast cells are pipetted into twenty four T150 flasks, each containing 40 ml of DMEM/F12 supplemented with 10% Bovine calf serum. The T-150 flasks are placed in a 5% CO₂ incubator at 37° C. Spent medium in the T-flasks is exchanged with 40 ml of fresh medium at day 4. At day 8, three T-flasks are removed from the incubator, cell morphology is noted, T-flasks are trysinized and the cells counted to get an average cell count. Twelve T-flasks are labeled as ‘Test Composition’ and twelve as ‘Control’. Spent medium is removed from all T-flasks. T-flasks are washed with 40 ml of PBS twice to remove traces of spent medium. 40 ml of the test composition is pipetted into the 12 T-flasks marked as ‘Test Composition’. 40 ml of basal cell culture medium (e.g. DMEM) is pipetted into the 12 T-flasks marked as ‘Control’. All flasks are placed in the incubator. At day 9, 10, 11 and 12, three T-flasks each labeled as ‘Test Article’ and ‘Control’ are removed, and flasks are observed for cell morphology. T-flasks are then trypsinized and cells counted.

Cell morphology for T-flasks containing the test composition is compared to control flasks and scored using a scale of 1-5. A statistically significant increase in maintenance of cell morphology over time for T-flasks containing the test composition versus the control demonstrates that the composition provides cell nutrition for improved cell maintenance, and thereby would be beneficial for skin cell care.

Cell growth rates for T-flasks containing the test composition is compared to control flasks. A 5-25% increase in cell growth rate for T-flasks containing test composition versus the control demonstrates that the composition provides enhancement of cell metabolism, and is thereby is beneficial for skin cell care.

Using the conventional definition with alpha=0.05, a result is statistically significant when the result would occur less than 10% of the time if the populations were really identical.

### EXAMPLE 9

This example describes an in vitro study demonstrating stimulation of extracellular matrix protein production and deposition of extracellular matrix proteins in fibroblasts according to the present invention. Concentrations of test composition permeating the skin, as determined in the percutaneous absorption model, are determined in this study. Phosphate buffer saline (PBS) is used as the control.

Human dermal fibroblast cells (obtained from a culture collection such as ATCC) are inoculated at 0.5 million cells in T-150 flasks containing 40 ml of DMEM/F12 supplemented with 10% bovine calf serum and grown in a 5% CO₂ incubator at 37° C. Confluent T-flask culture (usually grown for 12 days) are trypsinized, cells are counted and collected.

0.5 million human dermal fibroblast cells are pipetted into thirty T150 flasks, each containing 40 ml of DMEM/F12 supplemented with 10% bovine calf serum. The T-150 flasks are placed in a 5% CO₂ incubator at 37° C. Spent medium in the T-flasks is exchanged with 40 ml of fresh medium at day 4 and day 8. At day 12, all T-flasks are observed for confluency and only flasks that are 100% confluent are kept in the study. Three T-flasks are extracted with trypsin/EDTA/SDS mix to remove cells and extracellular matrix from the flasks. The extract is analyzed for quantitative measurement of collagen and GAG's to get an average baseline for ECM production. Twelve T-flasks are labeled with ‘Test Composition’ and twelve as ‘Control’. 40 ml of test composition is pipetted into the 12 T-flasks marked as ‘Test Composition’. 40 ml of basal cell culture medium (e.g. DMEM) is pipetted into the 12 T-flasks marked as ‘Control’. All flasks are placed in the incubator. At day 9, 10, 11 and 12, three T-flasks each labeled as ‘Test Composition’ and ‘Control’ are removed, flasks are extracted and analyzed for ECM production.

Total extracellular matrix (ECM) production and ECM production rates for T-flasks containing test composition is compared to Control flasks. A 5-50% increase in total ECM and ECM production rate for test composition T-flasks versus Control flasks demonstrates that the composition stimulates ECM production.

### EXAMPLE 10

This example describes an in vivo study demonstrating that the compositions of the invention moisturize skin in normal subjects with dry skin. This provides a measure of skin texture. Four different formulations of the invention are used as active compositions (Test Compositions A-D). Phosphate buffer saline is used as the negative control (Control).

Fifteen Caucasian female subjects, 30-50 years of age, are entered into the study and undergo a 7 day “wash out” phase in which the lower legs are washed with IVORY soap (Proctor & Gamble, Cincinnati, Ohio) twice a day and refrained from using any moisturization products on their lower legs.

After the seven-day “wash-out” period the subjects start the treatment with a 30 minute equilibration phase in an environment of 70+/−3° F. and a relative humidity of less than 30%. The lower legs of all subjects are evaluated for dryness to qualify for the study. Subjects with no dryness are eliminated from the study group. The subjects enrolled in the study have six 4×6 cm squares marked on the outer aspect of the lower leg that had the driest skin. The test compositions are applied to the sites using a standard rotation to eliminate positional bias.

At time zero, approximately 20 μl of the test composition is applied to the appropriate test site with a latex finger cot. The sixth test site is kept as the untreated site. The subjects remain in the testing facility for the duration of the study in a quiet fashion without drinking excessive water, smoking or eating. Skin moisturization readings are taken in duplicate using a NOVA™-DPM meter 9003 at each test site approximately one, two, three and four hours after application of the test composition. D-SQUAME® discs (Cuderm Corp., Dallas, Tex.) are taken at baseline on the untreated site and at four-hour evaluation on all test sites to measure the amount of stratum corneum scaling. The D-SQUAME® discs are then imaged to determine skin moisture readings.

The desquamation index is known in the art as an indicator of dry skin flakiness. The greater the number, size, and thickness of dry skin flakes, the higher the desquamation index. A 15% or greater increase in skin moisturization reading and a 10% or greater decrease in the Desquamation Index for test compositions versus the control demonstrates that the compositions moisturize skin and thereby improve skin texture and provide a more youthful skin appearance. Various computer programs and instrumentation is available in the art to conveniently determine the desquamation index. The programs preferably apply a mask to the image to define a measurement area of 200 mm². A lookup table is applied to the image, which substitutes a new set of numeric values for the default gray scale so that ranges of gray levels are represented by single values. Each pixel is assigned to one of five arbitrary thickness levels of the corneocyte clusters. Transformations can be used to calculate the number of pixels in each thickness group as a percent value as well as the total area occupied with cells. The percentage area occupied by corneocytes is also determined. These two functions are integrated to yield the desquamation index according to the following formula:

$$D . I . = \frac{2 A + \begin{matrix}5 \\ n = 1\end{matrix} Tn * ( n - 1 )}{6}$$

where D.I. is the desquamation index, A is the percent area covered by corneocytes, Tn is the percentage of corneocytes in relations to thickness, and n is the thickness level (1-5).

### EXAMPLE 11

This example describes an in vivo study designed to evaluate the compositions for effectiveness in rejuvenating the skin, by reducing the occurrence of facial lines in a double-blind vehicle controlled study. A formulation of the invention is used as an active composition (Test Composition). Phosphate buffer saline is used as the vehicle control (Control).

Thirty females ages 35-75, selected for the study based on good general health are entered into the in the study and undergo a 7 day “wash out” period in which they refrain from using all facial moisturizing products (i.e. soaps, creams, lotions, gels). The subjects are given a non-moisturizing glycerin soap (Neutrogena Corp., Los Angeles, Calif.) to use for their daily cleansing of the face throughout the “wash-out and treatment” phases. The subjects are allowed to use their regular make-up during the wash-out period and for the duration of the study.

On day 1 after the “wash-out” period, 35 mm color photographs are taken (with a 70-300 mm macro lens) of each side of the face followed by silicone replicas from the crow's feet area. The procedure for taking replicas is as follows: two or three drops of catalyzer is added to the SILFLO® resin (Silflo, Flexico Developments Ltd., Potters Bar, England) and is rapidly mixed. The paste is then immediately applied to the skin surface. After two or three minutes, the replica is gently removed from the skin and moisture readings recorded from a NOVA™ DPM meter 9003, or equivalent instrument that measures moisture content of skin by electric conductance.

Additional replicas are taken from the cheek area (duplicate reading from each cheek) to determine the level of the skin surface moisture. Also in the cheek area D-SQUAME® tape is used to take specimens of the outer surface of the stratum corneum in order to measure the effect of the test composition on reducing skin roughness. Each subject is given product to use at home. The assignment of Test Composition and Control is alternated from subject to subject. Each subject is given a diary to take home to record the time of each application as well as to report any sensory or visual irritation. The subjects return after four and eight week intervals to have their diaries checked and to receive more Test Composition or Control as necessary. The subjects return to the testing facility after a total of 12 weeks of treatment for final silicone replicas, photographs, NOVA™ readings, and to have D-SQUAME® tape applied to the skin.

The D-SQUAME® tape and silicone replicas are analyzed by electronic imaging and image analysis to determine the desquamation index. A statistically significant increase in the skin moisturization reading and a decrease in the Desquamation Index for test composition subjects versus control subjects demonstrates that the compositions increase skin moisturization and thereby improve skin texture and enhance a youthful skin appearance. A reduction in the number of fine lines assessed by image analysis of the silicone replicas of subjects using test composition versus Control demonstrates that the compositions reduce fine lines and wrinkles, and thereby strengthen the skin structure.

### EXAMPLE 12

This example describes an in vivo study designed to illustrate the present compositions' effectiveness in wound healing in human subjects in a double-blind vehicle controlled study. A composition formulated for wound healing application is used as a the test composition.

Wounds are created in the forearms of eight human volunteers using a keratome with a setting of approximately 0.3 mm. A measurement of the width of the wound is taken (in mm). Treatment is begun immediately after wounding and twice a day for the next four days. Photographs of the wound and clinical observations are conducted daily.

On the second and fourth days after wounding, a measurement of the width of the wound is taken along with a 2 mm biopsy. The biopsy is processed according to standard procedures and the tissue sections are stained with H&E. A dermal pathologist evaluates the histological sections for vascularization, inflammation and epithelialization. Wound healing capabilities of the test composition is demonstrated by any of the following criteria: a 10-30% reduction in wound width measurement, an increase in vascularization of the wound, a reduction in inflammation, or an increase in epithelialization using the composition versus the control.

### EXAMPLE 13

This example describes an in vivo study designed to demonstrate the present invention's effectiveness in increasing hair growth on the scalp of human subjects, or in decreasing the rate of hair loss on the scalp. The study is a double-blind vehicle controlled study. A composition formulated for hair growth application is used as an active composition.

Approximately 30 men, ages 18 to 40 with alopecia androgenetica as evidenced by frontal/parietal hair thinning, as defined by the Hamilton Scale as Type III or IV, are enrolled in the study.

A representative site on the thinning frontal/parietal scalp is chosen as the treatment site. The site is marked using permanent ink at the four corners. The hair is carefully clipped from the test site, counted and weighed. After six weeks the process is repeated and the treatment is begun.

The subjects return to the test facility every six weeks for six months to have the hair in the treatment site clipped and weighed. A 5-25% increase in weight of hair clippings or hair count among test composition subjects versus control subjects demonstrates that the compositions increase hair growth on the scalp, and that the compositions will decrease the rate of hair loss on the scalp.

The following are additional examples and embodiments of the invention:

In one embodiment the composition contains at least one cell growth factor selected from epidermal growth factor (EGF), fibroblast growth factor (FGF), insulin-like growth factor (IGF), granulocyte colony stimulating factor (GCSF), granulocyte macrophage colony stimulating factor (GMCSF), platelet derived growth factor (PDGF), keratinocyte growth factor (KGF), tissue growth factor-α (TGF-α); and

at least one cell growth enhancer selected from erythropoietin, hematopoietin, prostaglandin, human growth hormone, VEGF, acidic FGF, TGF-α, PDGF, fibronectin, vitronectin, thrombospondin, laminin, tenasin.

In one embodiment the carbohydrates are monosaccharides, disaccharides, or polysaccharides,

the lipid, phospholipid, and fatty acid are selected from linoleic acid, lipoic acid, cholesterol, and oleic acid and derivatives thereof; and

the essential and non essential amino acids are selected from L-Alanine, L-Arginine.HCl, L-Asparagine, L-Aspartic Acid, L-Cystine.HCl, L-Cysteine-2HCl, L-Glutamic Acid, L-Glutamine, Glycine, L-Histidine.HCl, L-Isoleucine, L-Leucine, L-Lysine.HCl, L-Methionine, L-Phenylalanine, L-Proline, L-Serine, L-Threonine, L-Tryptophan, L-Tyrosine, L-Valine and derivatives thereof;

the inorganic salts, minerals, and trace metals are selected from copper sulfate, ferric nitrate, ferrous sulfate, potassium chloride, sodium chloride, sodium phosphate monobasic and dibasic, zinc sulfate, sodium acetate (anhydrous), sodium pyruvate, manganese sulfate, ammonium molybedate, nickel chloride, tin chloride, potassium phosphate monobasic and dibasic, sodium silicate, sodium selenite, and derivatives thereof;

the vitamins are selected from D-Biotin, Choline Chloride, Folic Acid, myo-Inositol, Niacinamide, D-Pantothenic Acid, calcium salt, Pyridoxal.HCl, Pyridoxine.HCl, Riboflavin, Thiamine.HCl, Vitamin B-12 and derivatives and analogs thereof; and

the nucleoside is thymidine or and derivatives thereof;

the purine, and pyrimidine are selected from Hypoxanthine, Adenine.HCl and derivatives thereof;

the buffer is selected from HEPES, sodium bicarbonate and analogs and derivatives thereof; and

the other nutrients are selected from putrescine, protein or protein extract, peptide, peptone, serum or serum substitute, natural extract, plant or animal derived component.

In one embodiment the fibrous protein is selected from the group consisting of: collagens, elastins, α-keratins, and fibroin; and the adhesion molecules are selected from the group consisting of: cadherins, immunoglobulin superfamily, integrins, proteoglycans, and selectins and analogs thereof.

In one embodiment the composition contains at least one growth factor selected from the group consisting of: TGF-beta, basic FGF, VEGF, other growth factors;

the one or more stimulators of extra-cellular matrix protein production are selected from the group consisting of: sodium ascorbate, hyaluronic acid, tretinoin (RETIN-A®) and analogs, Heparin-Sodium; and

the adhesion proteins are selected from the group consisting of: Fibronectin, Vitronectin, Laminin, thrombospondin, tenasin, stress proteins such as GR78, and short chain peptides.

In one embodiment the at least one or more hydrophobic penetration enhancers of the composition are selected from the group consisting of: lipids and lipoproteins;

at least one or more hydrophilic penetration enhancers are selected from the group consisting of: detergents, fatty acids, fatty alcohol, alcohols, glycols; and

at least one or more hydrophobic and hydrophilic penetration enhancers are selected from the group consisting of hydrophobic and hydrophilic block copolymers.

In one embodiment the composition includes a transdermal delivery vehicle selected from the group consisting of: liposomes, micelles, emulsions, micro-emulsions, an aqueous solution, a non-aqueous solution; and

a transdermal delivery device selected from the group consisting of: an electrical pump, a transdermal patch, an occlusive pad, an adhesive or non-adhesive bandage, a gel, and a film.

In one embodiment the lipids of the composition comprise an oil selected from the group consisting of: mineral oil, herbal oil, animal oil, synthetic oil, natural oil, citral oil, almond oil, coconut oil, linseed oil, camphor, menthane, and a terpene;

the lipoproteins are selected from the group consisting of: shea butter, cacao butter, triglycerides, a plant lipoprotein, wheat protein, soy protein, an animal lipoprotein, and milk;

the detergent is selected from the group consisting of: an ionic or nonionic surfactant, Polysorbate 80, Polyoxyethylenesorbitan Monooleate, and sodium dodecylsulfate (SDS);

the fatty acids are selected from the group consisting of: linoleic acid, oleic acid;

the fatty alcohol is selected from the group consisting of: Cetyl alcohol (natural and iso-), Oleyl alcohol, Cetyl-stearyl alcohol, Octyl alcohol, Decyl alcohol, Ricinol alcohol, Lauryl alcohol, Strearyl alcohol, Lauro-myristyl alcohol, Tallow alcohol;

the alcohols are selected from the group consisting of: methanol, ethanol, propanol, isopropanol, and analogs;

the glycols are selected from the group consisting of: propylene glycol, butylene glycol and analogs.

In one embodiment the composition contains at least one growth enhancer selected from the group consisting of: epidermal growth factor (EGF), fibroblast growth factor (FGF), insulin-like growth factor (IGF), granulocyte colony stimulating factor (GCSF), granulocyte macrophage colony stimulating factor (GMCSF), platelet derived growth factor (PDGF), keratinocyte growth factor (KGF), and tissue growth factor-α (TGF-α);

at least one growth enhancer selected from the group consisting of: cytokines, regulatory factors, angiogenic factors, and adhesion proteins; and

at least one stimulator of cell growth enhancers selected from the group consisting of: hyaluronic acid, sodium hyaluronate, ascorbic acid, sodium ascorbate, and amphiregulin;

a mixture of nutrients in an amount effective to support log phase growth of skin cells comprising at least one carbohydrate, at least one lipid, at least one phospholipid, at least one fatty acid, at least one essential or non essential amino acid, at least one inorganic salt, at least one mineral, at least one trace metal, at least one vitamin, at least one nucleoside, at least one purine, at least one pyrimidine, and at least one buffer;

at least one cell protector in an amount effective to protect growing cells and maintain enhanced cellular activity selected from the group consisting of: insulin, transferrin, selenium, and analogs and derivatives thereof;

at least one antioxidant in an amount effective to protect rejuvenated cells from damage selected from the group consisting of: vitamins, coenzyme Q10, cysteine, glutathione, a thiol or analogs and derivatives thereof;

at least one extra-cellular matrix protein selected from the group consisting of: a fibrous protein and an adhesion molecule;

stimulators of extra-cellular matrix protein production comprising one or more growth factors, one or more chemical stimulators, and one or more adhesion proteins;

penetration enhancers comprising one or more hydrophobic enhancers, one or more hydrophilic enhancers, and one or more hydrophobic and hydrophilic copolymers.

In one aspect the invention provides methods for repairing mammalian skin. The methods involve contacting the skin with a composition of claim 1 for a period of time sufficient for the cell growth enhancers, stimulants of growth enhancers, nutrients, cell protectors, antioxidants, extracellular matrix proteins, and stimulators of extracellular matrix to permeate the skin in an amount effective to repair the skin.

In various embodiments the repair includes the rejuvenation of the skin, or the healing of a wound, or the reduction in fine lines and wrinkles of the treated skin of 10% or more. The mammalian skin can include hair follicles. Growth of hair from the hair follicles can be increased by 10% or more using the methods. In some embodiments the wound is a sunburn or a topical abrasion.

The methods can also include applying the composition as a coating on a medical or surgical device, such as a suture, an implant, a homeostatic plug, a wound dressing, a gauze bandage, and a pad.

In one embodiment the method for repairing mammalian skin involves contacting the skin with a composition of the invention for a period of time sufficient for the cell growth enhancers, nutrients, extracellular matrix proteins, and stimulators of extracellular matrix to permeate mammalian skin in an amount effective to repair the skin.

In another embodiment the invention privides a method for increasing hair growth on the skin of the mammalian scalp. The methods involve contacting the skin of the mammalian scalp with a composition of the invention for a period of time sufficient for the cell growth enhancers, nutrients, extracellular matrix proteins, and stimulators of extracellular matrix to permeate the mammalian skin in an amount effective to increase hair growth.

The invention illustratively described herein may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A composition for skin repair comprising:
at least one cell growth enhancer and at least one stimulator of cell growth enhancers in an amount effective to increase the growth rate of skin cells;
a combination of nutrients in an amount effective to support log phase growth of skin cells;
at least one cell protector in an amount effective to protect growing cells and maintain enhanced cellular activity;
at least one antioxidant in an amount effective to protect rejuvenated cells from damage;
one or more extra-cellular matrix proteins in an amount effective to improve skin structure; and
one or more stimulators of extra-cellular matrix protein production in an amount effective to increase the production of extra-cellular matrix proteins in the skin; and
a mixture of penetration enhancers in an amount effective to allow penetration of the the dermal layer by the cell growth enhancers, stimulators of cell growth enhancers, nutrients, cell protectors, antioxidants, extracellular matrix proteins, and stimulators of extracellular matrix protein production in an amount effective to promote skin repair; and
a biologically acceptable carrier.

2. The composition of claim 1 wherein:
at least one cell growth enhancer is selected from the group consisting of cell growth factors;
at least one growth enhancer is selected from the group consisting of: cytokines, regulatory factors, angiogenic factors, and adhesion proteins;
at least one stimulator of cell growth enhancers is selected from the group consisting of: hyaluronic acid, sodium hyaluronate, ascorbic acid, sodium ascorbate and amphiregulin;
the mixture of nutrients effective to support log phase growth of skin cells are selected from the group consisting of: carbohydrates, lipids, phospholipids, fatty acids, essential and non essential amino acids, inorganic salts, minerals, and trace metals, vitamins, nucleosides, purines, and pyrimidines, buffers, other nutrients and, analogs and derivatives thereof;
at least one cell protector is selected from the group consisting of: insulin, transferrin, selenium, and analogs and derivatives thereof;
at least one antioxidant is selected from the group consisting of: Vitamins E, Vitamin C, and Vitamin K, beta-carotene, coenzyme Q 0, cysteine, glutathione, a thiol or analogs and derivatives thereof;
at least one extra-cellular matrix protein is selected from each of the categories consisting of: fibrous proteins, and adhesion molecules;
at least one stimulator of extra-cellular matrix protein production is selected from each of the categories consisting of: growth factors, chemical stimulators and adhesion proteins; and
at least one penetration enhancer is selected from each of the groups consisting of: hydrophobic enhancers, hydrophilic enhancers, and hydrophobic and hydrophilic copolymers.

3. The composition of claim 2 wherein
at least one cell growth factor is selected from the group consisting of: epidermal growth factor (EGF), fibroblast growth factor (FGF), insulin-like growth factor (IGF), granulocyte colony stimulating factor (GCSF), granulocyte macrophage colony stimulating factor (GMCSF), platelet derived growth factor (PDGF), keratinocyte growth factor (KGF), tissue growth factor-α (TGF-α);
at least one cell growth enhancer is selected from the group consisting of: erythropoietin, hematopoietin, prostaglandin, human growth hormone, VEGF, acidic FGF, TGF-α, PDGF, fibronectin, vitronectin, thrombospondin, laminin, tenasin.

4. The composition of claim 2 wherein
the carbohydrates are selected from the group consisting of: monosaccharides, disaccharides, and polysaccharides,
the lipid, phospholipid, and fatty acid are selected from the group consisting of: linoleic acid, lipoic acid, cholesterol, and oleic acid and derivatives thereof; and
the essential and non essential amino acids are selected from the group consisting of: L-Alanine, L-Arginine.HCl, L-Asparagine, L-Aspartic Acid, L-Cystine.HCl, L-Cysteine.2HCl, L-Glutamic Acid, L-Glutamine, Glycine, L-Histidine.HCl, L-Isoleucine, L-Leucine, L-Lysine.HCl, L-Methionine, L-Phenylalanine, L-Proline, L-Serine, L-Threonine, L-Tryptophan, L-Tyrosine, L-Valine and derivatives thereof;
the inorganic salts, minerals, and trace metals are selected from the group consisting of: copper sulfate, ferric nitrate, ferrous sulfate, potassium chloride, sodium chloride, sodium phosphate monobasic and dibasic, zinc sulfate, sodium acetate (anhydrous), sodium pyruvate, manganese sulfate, ammonium molybedate, nickel chloride, tin chloride, potassium phosphate monobasic and dibasic, sodium silicate, sodium selenite, and derivatives thereof;
the vitamins are selected from the group consisting of: D-Biotin, Choline Chloride, Folic Acid, myo-Inositol, Niacinamide, D-Pantothenic Acid, calcium salt, Pyridoxal.HCl, Pyridoxine.HCl, Riboflavin, Thiamine.HCl, Vitamin B-12 and derivatives and analogs thereof; and
the nucleoside is thymidine or and derivatives thereof;
the purine, and pyrimidine are selected from the group consisting of: Hypoxanthine, Adenine.HCl and derivatives thereof;
the buffer is selected from the group consisting of: HEPES, sodium bicarbonate and analogs and derivatives thereof; and
the other nutrients are selected from the group consisting of: putrescine, protein or protein extract, peptide, peptone, serum or serum substitute, natural extract, plant or animal derived component.

5. The composition of claim 2 wherein
the fibrous protein is selected from the group consisting of: collagens, elastins, α-keratins, and fibroin;
the adhesion molecules are selected from the group consisting of: cadherins, immunoglobulin superfamily, integrins, proteoglycans, and selectins and analogs thereof.

6. The composition of claim 2 wherein,
at least one (?) or more growth factors are selected from the group consisting of: TGF-beta, basic FGF, VEGF, other growth factors;
the one or more stimulators of extra-cellular matrix protein production are selected from the group consisting of: sodium ascorbate, hyaluronic acid, tretinoin (RETIN-A®) and analogs, Heparin-Sodium; and
the adhesion proteins are selected from the group consisting of: Fibronectin, Vitronectin, Laminin, thrombospondin, tenasin, stress proteins such as GR78, and short chain peptides.

7. The composition of claim 2 wherein
at least one or more hydrophobic penetration enhancers are selected from the group consisting of: lipids and lipoproteins;
at least one or more hydrophilic penetration enhancers are selected from the group consisting of: detergents, fatty acids, fatty alcohol, alcohols, glycols; and
at least one or more hydrophobic and hydrophilic penetration enhancers are selected from the group consisting of hydrophobic and hydrophilic block copolymers.

8. The composition of claim 1 further comprising
a transdermal delivery vehicle selected from the group consisting of: liposomes, micelles, emulsions, micro-emulsions, an aqueous solution, a non-aqueous solution; and
a transdermal delivery device selected from the group consisting of: an electrical pump, a transdermal patch, an occlusive pad, an adhesive or non-adhesive bandage, a gel, and a film.

9. A composition of claim 7 wherein:
the lipids comprise an oil selected from the group consisting of: mineral oil, herbal oil, animal oil, synthetic oil, natural oil, citral oil, almond oil, coconut oil, linseed oil, camphor, menthane, and a terpene;
the lipoproteins are selected from the group consisting of: shea butter, cacao butter, triglycerides, a plant lipoprotein, wheat protein, soy protein, an animal lipoprotein, and milk;
the detergent is selected from the group consisting of: an ionic or nonionic surfactant, Polysorbate 80, Polyoxyethylenesorbitan Monooleate, and sodium dodecylsulfate (SDS);
the fatty acids are selected from the group consisting of: linoleic acid, oleic acid;
the fatty alcohol is selected from the group consisting of: Cetyl alcohol (natural and iso-), Oleyl alcohol, Cetyl-stearyl alcohol, Octyl alcohol, Decyl alcohol, Ricinol alcohol, Lauryl alcohol, Strearyl alcohol, Lauro-myristyl alcohol, Tallow alcohol;
the alcohols are selected from the group consisting of: methanol, ethanol, propanol, isopropanol, and analogs;
the glycols are selected from the group consisting of: propylene glycol, butylene glycol and analogs.

10. A composition for skin repair comprising:
at least one growth enhancer is selected from the group consisting of: epidermal growth factor (EGF), fibroblast growth factor (FGF), insulin-like growth factor (IGF), granulocyte colony stimulating factor (GCSF), granulocyte macrophage colony stimulating factor (GMCSF), platelet derived growth factor (PDGF), keratinocyte growth factor (KGF), and tissue growth factor-α (TGF-α);
at least one growth enhancer selected from the group consisting of: cytokines, regulatory factors, angiogenic factors, and adhesion proteins; and
at least one stimulator of cell growth enhancers selected from the group consisting of: hyaluronic acid, sodium hyaluronate, ascorbic acid, sodium ascorbate, and amphiregulin;
a mixture of nutrients in an amount effective to support log phase growth of skin cells comprising at least one carbohydrate, at least one lipid, at least one phospholipid, at least one fatty acid, at least one essential or non essential amino acid, at least one inorganic salt, at least one mineral, at least one trace metal, at least one vitamin, at least one nucleoside, at least one purine, at least one pyrimidine, and at least one buffer;
at least one cell protector in an amount effective to protect growing cells and maintain enhanced cellular activity selected from the group consisting of: insulin, transferrin, selenium, and analogs and derivatives thereof;
at least one antioxidant in an amount effective to protect rejuvenated cells from damage selected from the group consisting of: vitamins, coenzyme Q10, cysteine, glutathione, a thiol or analogs and derivatives thereof;
at least one extra-cellular matrix protein selected from the group consisting of: a fibrous protein and an adhesion molecule;
stimulators of extra-cellular matrix protein production comprising one or more growth factors, one or more chemical stimulators, and one or more adhesion proteins;
penetration enhancers comprising one or more hydrophobic enhancers, one or more hydrophilic enhancers, and one or more hydrophobic and hydrophilic copolymers.

11. A method for repairing mammalian skin comprising:
contacting the skin with a composition of claim 1 for a period of time sufficient for the cell growth enhancers, stimulants of growth enhancers, nutrients, cell protectors, antioxidants, extracellular matrix proteins, and stimulators of extracellular matrix to permeate the skin in an amount effective to repair the skin.

12. The method of claim 11 wherein the repair comprises the rejuvenation of the skin.

13. The method of claim 11 wherein the repair comprises the healing of a wound.

14. The method of claim 11 wherein the repair comprises a reduction in fine lines and wrinkles of the treated skin of 10% or more.

15. The method of claim 11 wherein the mammalian skin comprises hair follicles.

16. The method of claim 15 wherein growth of hair from the hair follicles increases by 10% or more.

17. The method of claim 13 wherein the wound is a sunburn or a topical abrasion.

18. The method of claim 11 further comprising:
applying the composition as a coating on a medical or surgical device selected from the group consisting of: a suture, an implant, a homeostatic plug, a wound dressing, a gauze bandage, and a pad.

19. A method for repairing mammalian skin
contacting the skin with a composition of claim 10 for a period of time sufficient for the cell growth enhancers, nutrients, extracellular matrix proteins, and stimulators of extracellular matrix to permeate mammalian skin in an amount effective to repair the skin.

20. A method for increasing hair growth on the skin of the mammalian scalp comprising:
contacting the skin of the mammalian scalp with a composition of claim 10 for a period of time sufficient for the cell growth enhancers, nutrients, extracellular matrix proteins, and stimulators of extracellular matrix to permeate the mammalian skin in an amount effective to increase hair growth.
